(19) United States
(12) Patent Application Publication
(10) Pub. No.: US 2018/0317842 A1
(43) Pub. Date: Nov. 8, 2018

(54) A CABLE SYSTEM WITH SENSOR

(71) Applicant: TOBB EKONOMI VE TEKNOLOJI UNIVERSITESI, Cankaya/Ankara (TR)

(72) Inventors: Tolga Tolunay, Yenimahalle/Ankara (TR); Teyfik Demir, Cankaya/Ankara (TR); Hilmi Volkan Demir, Bilkent/Ankara (TR)

(21) Appl. No.: 15/769,895

(22) PCT Filed: Oct. 24, 2016
(86) PCT No.: PCT/TR2016/000146

§ 371 Date: Apr. 20, 2018

(30) Foreign Application Priority Data
Oct. 26, 2015 (TR) 2015/13361

(51) Int. Cl. A61B 5/00; A61B 17/82; A61B 17/80

(52) U.S. Cl. CPC A61B 5/4851; A61B 2017/00221; A61B 17/8061; A61B 17/82

(57) ABSTRACT

This invention is related to a cable system (1) having a sensor, integrated onto the implant, which is attached to the femur bone and which envelopes the femur bone, enabling to measure the force applied on the femur bone by implants providing the required force, and which also carries out the enveloping force that is required in order for the implant to be used efficiently.

### TECHNICAL FIELD

This invention is related to a cable system having a sensor, integrated onto the implant, which is attached to the femur bone and which envelopes the femur bone, enabling to measure the force applied on the femur bone by implants providing the required force, and which also carries out the winding force that is required in order for the implant to be used efficiently.

### PRIOR ART

The hip is one of the largest load bearing joints of our body. The hip joint is formed from the joining of the round end of the thigh bone with the hip bone. The surfaces that come together are coated with cartilage and they obtain their momentum from strong muscles. Different types of prosthesis are used against damages that may occur in said bones, and it is aimed for the femur bone to be strengthened and to be in a state that shall not affect the daily life of a person. However when prosthesis are fixed to the bone, it is difficult to obtain the desired precision, and the bone into which the implant is inserted into can be damaged. If the cable of the supplementary prosthesis having a cable which is one of the prosthesis types is tightened too much, this may damage the bone or may even break or fracture the bone. On the other hand when the cable is not tightened sufficiently, the prosthesis cannot provide its necessary duty and this may necessitate re-operations.

### AIMS OF THE INVENTION

The aim of the invention is to develop a cable system having a sensor, that is attached to the femur bone and which provides the required force by enveloping the femur bone and enables to measure the force applied on the femur bone.

Another aim of this invention is to provide a cable system having a sensor which can measure the implant force that can be endured by the femur bone.

Another aim of this invention is to provide a cable system having a sensor which can measure the winding force that is require in order for the implant to be used efficiently.

Another aim of this invention is to provide a cable system having a sensor that can be integrated onto the implant.

### DETAILED DESCRIPTION OF THE INVENTION

The cable system having a sensor provided in order to reach the aim of the invention has been shown in the attached figures, wherein said figures illustrate the following;

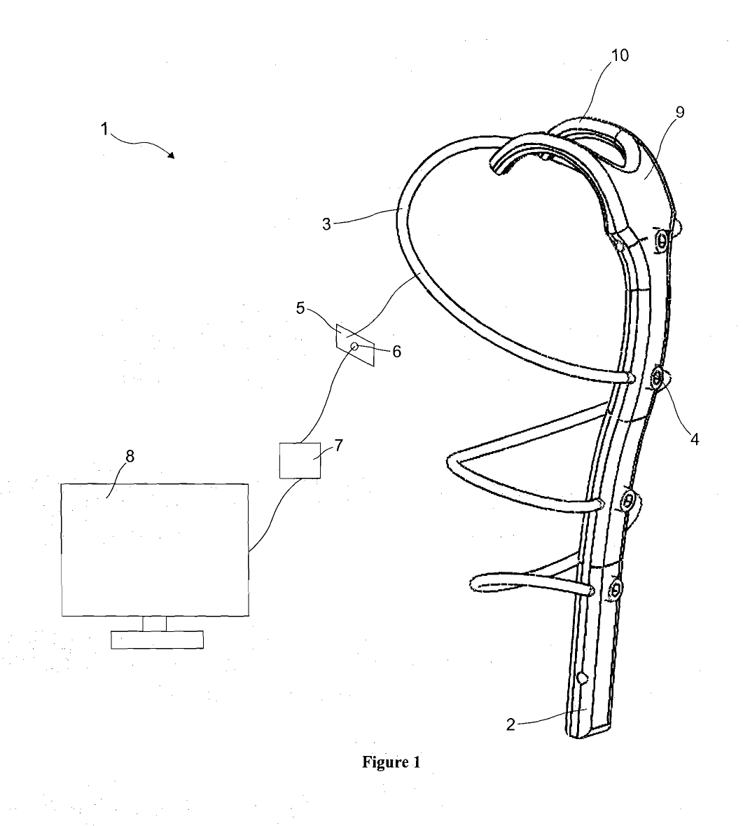

FIG. 1. Shows the perspective schematic view of the cable system having a sensor.

The parts in the figures are numbered individually and the references corresponding to the related part are listed below.
1. Cable system with sensor
2. Main Body
3. Tightening cable
4. Adjustment hole
5. Sensor
6. Transmitter
7. Receiver
8. Monitor
9. Upper body
10. Bone holder

The cable system (1) having a sensor, integrated onto the implant, which is attached to the femur bone and which envelopes the femur bone, enabling to measure the force applied on the femur bone by implants providing the required force, and which also carries out the enveloping force that is required in order for the implant to be used efficiently, basically comprises;
at least a main body (2),
At least a tightening cable (3), which extends along the main Body (2) and which enables the implant to envelope the bone,
At least an adjustment hole (4) located in the main body (2) which enables the winding of the tightening cable (3) around the bone to be wound with force,
At least a sensor (5) positioned within the tightening cable (3) in order to measure the force applied onto the tightening cable (3), the force direction, displacement, displacement direction, deformation, deformation direction and all other similar data,
at least a transmitter (6) located within the sensor (5) which transmits the information measured in vivo by the sensor (5) to the outer environment,
at least a receiver (7) which receives information provided by the transmitter (6),
at least a monitor (8) which enables to display the information obtained from the receiver (7)
at least an upper body (9) along the main body (2) which enables the main body (2) to be attached to the bone by means of its concave geometry,
at least a bone holder (10) located on the main body (9) which enables fitting to the bone,

The cable system (1) with sensor subject to the invention according to the preferred embodiment of the invention comprises a main body (2) and an upper body (9), The tightening cable (3) located on the main body moves along the main body (2) and enables the implant to precisely fit the bone, The adjustment holes (4) located on the main body (2) are aligned preferably along the path from which the tightening cable (3) passes from and by this means the desired level of tightening can be carried out. The tightening cable (3) applies the desired force on the bone by means of the adjustment holes. By this means the bone is both prevented from being damages and it is enabled for the implant to be used more efficiently. The adjustment holes (4) are formed to ve suitable to the required apparatus and the tightening cable (3) is also adjusted with the desired precision by means of said adjustment holes. A sensor (5) is positioned within the tightening cable (3) in order to measure the force applied onto the tightening cable (3), the force direction, displacement, displacement direction, deformation, deformation direction and all other similar data. The desired precision is provided by means of this sensor (5) and the bone is prevented from being damaged. The implant can be fitted into the bone having the desired force by means of the sensor (5) data that is read. A transmitter (6) located on the sensor (5) transmits the information that has been measured to an external source. The transmitter (6) can selectively use the desired protocol and communication structure. A receiver (7) is provided which obtains the information transmitted by the transmitter (6) to the external source. This receiver (7) transmits the information it has received to the monitor (8). The monitor (8) can either show the information or can carry out an analysis process in order to provide the information that is required by the user. Besides these, at least an upper body (9) along the main body (2) which enables the main body (2) to be attached to the bone by means of its concave geometry is provided. The upper body (9) has a structure that can be shaped in accordance with the curve of the bone and by this means it is enabled for the implant to precisely fit the bone. Moreover an exact hold is provided between the bone and the implant by means of the bone holder (10) located along the upper body (9), and therefore the implant is prevented from moving.

In an embodiment of the invention the sensor (5) provided on the cable system (1) with sensor (5) can not only submit the information by means of the transmitter (6) but it can also transmit the information directly.

The force obtained by means of the cable system (1) with a sensor subject to the invention according to another embodiment of the invention, and the force direction, displacement, displacement direction, deformation, deformation direction and all other similar data, can be both viewed and analyzed from the monitor (8). Such information analyzed, can also be supplied in printed form.

1. The cable system (1) having a sensor, integrated onto the implant, which is attached to the femur bone and which envelopes the femur bone, enabling to measure the force applied on the femur bone by implants providing the required force, and which also carries out the enveloping force that is required in order for the implant to be used efficiently is characterized in that it basically comprises;
at least a main body (2),
at least a tightening cable (3), which extends along the main Body (2) and which enables the implant to envelope the bone,
at least an adjustment hole (4) located in the main body (2) which enables the winding of the tightening cable (3) around the bone to be wound with force,
at least a sensor (5) positioned within the tightening cable (3) in order to measure the force applied onto the tightening cable (3), the force direction, displacement, displacement direction, deformation, deformation direction and all other similar data.

2. A cable system (1) with sensor according to claim 1 characterized by at least a transmitter (6) located within the sensor (5) which transmits the information measured in vivo by the sensor (5) to the outer environment.

3. A cable system (1) with sensor according to claim 1 characterized by at least a receiver (7) which receives information provided by the transmitter (6).

4. A cable system (1) with sensor according to claim 1 characterized by at least a monitor (8) which enables to display the information obtained from the receiver (7)

5. A cable system (1) with sensor according to claim 1 characterized by at least an upper body (9) along the main body (2) which enables the main body (2) to be attached to the bone by means of its concave geometry.

6. A cable system (1) with sensor according to claim 1 characterized by at least a bone holder (10) located on the main body (9) which enables fitting to the bone.

7. A cable system (1) with sensor according to claim 1 characterized by a tightening cable (3) which is located along the main body (2) and which enables the implant to be fitted exactly to the bone.

8. A cable system (1) with sensor according to claim 1 characterized by at least a tightening cable (3) which prevents the bone from being damaged and which enables the implant to be used efficiently.

9. A cable system (1) with sensor according to claim 1 characterized by an adjustment hole (4) which is preferably placed along the path of the tightening cable (3) thereby enabling a tightening process at the desired level.

10. A cable system (1) with sensor according to claim 1 characterized by an adjustment hole (4) which enables the desired amount of force to be applied on the bone by the tightening cable (3).

11. A cable system (1) with sensor according to claim 1 characterized by an adjustment hole (4) that is formed to be suitable to an apparatus.

12. A cable system (1) with sensor according to claim 1 characterized by an adjustment hole (4) which enables the tightening cable (3) to be adjusted with precision.

13. A cable system (1) with sensor according to claim 1 characterized by a sensor (5) which provides the desired precision and which prevents the bone from being damaged by this means.

14. A cable system (1) with sensor according to claim 1 characterized by a sensor (5) which enables the implant to be fitted with the desired implant force density to the bone.

15. A cable system (1) with sensor according to claim 1 characterized by a receiver (6) which can use the desired protocol and communication structure.

16. A cable system (1) with sensor according to claim 1 characterized by at least an upper body (9) along the main body (2) which enables the main body (2) to be attached to the bone by means of its concave geometry.

17. A cable system (1) with sensor according to claim 1 characterized by a curved upper body (9) which can be shaped and therefore which enables the implant to be fitted exactly to the bone.

18. A cable system (1) with sensor according to claim 1 characterized by at least a bone holder (10) located along the main body (9) which enables exact fitting between the implant and the bone.

19. A cable system (1) with sensor according to claim 1 characterized by a receiver (5) which can directly transmit information.
